(19) United States
(12) Patent Application Publication
(10) Pub. No.: US 2014/0206951 A1
(43) Pub. Date: Jul. 24, 2014

(54) Arthroscope Sheath System With Sensors

(71) Applicant: Arthrex, Inc., Naples, FL (US)

(72) Inventors: Tom Deppmeier, Santa Ynez, CA (US); Craig Speier, Santa Barbara, CA (US); David D'Alfonso, Gaviota, CA (US)

(21) Appl. No.: 14/163,742

(22) Filed: Jan. 24, 2014

Related U.S. Application Data
(60) Provisional application No. 61/756,134, filed on Jan. 24, 2013.

(51) Int. Cl. A61B 1/317; A61B 5/01; A61B 5/03

(52) U.S. Cl. CPC A61B 1/317; A61B 5/036; A61B 5/01
USPC 600/301; 600/300; 600/561; 600/549

(57) ABSTRACT

An arthroscopic surgery sheath system having: a body for insertion of an instrument; an insertion portion coupled to the body; a valve coupled to the insertion portion; a tube coupled to the valve; and a microelectromechanical sensor positioned inside the valve or the tube for measurement of at least one characteristic of a fluid inside of the tube; wherein the sensor is configured to transmit measurement information.

### CROSS REFERENCE TO RELATED APPLICATION

This application claims priority of U.S. Provisional Patent Application No. 61/756,134, filed on Jan. 24, 2013, entitled ARTHROSCOPE SHEATH SYSTEM WITH SENSORS, the entire contents of which are hereby incorporated herein by reference.

### BACKGROUND OF THE INVENTION

The present invention relates to devices used in arthroscopic surgery and, more particularly, to fluid inflow and outflow sheath systems having sensors for use in arthroscopic surgery.

In endoscopic surgical procedures and, in particular, in arthroscopic procedures, there is often a need to know the pressure and/or temperature at the surgical work site. For example, in some arthroscopic surgical procedures a joint being operated on is subjected to irrigating fluid pressure in order to distend the joint to provide an adequate work space and in order to keep the joint free of debris while enhancing visibility during the procedure. An outflow channel is provided to maintain fluid movement through the work site.

The fluid may be pressurized by a pump which forces the fluid into the work site, or it may be pressurized by gravity. While some pressure is necessary, an excessive amount of pressure may cause extravasation into surrounding tissue or otherwise injure the patient. Consequently, pressure sensing devices are desirable during many arthroscopic surgical procedures to control the fluid pressure being supplied to the work site.

However, prior art sensing devices typically suffer from one or more of the following shortcomings: the sensors are wired and require physical connections, the sensors are not suitable for use in disposable components, the sensors require the use of separate or larger and more invasive tubing. Additionally some sensors require additional channels independent of the regular fluid inflow/outflow channels which increases the size of the sheath making it more difficult to manipulate, such as in a joint, and increasing the risk of injury to the surgical site. Additionally, some sensors are positioned at locations within a fluid system, such as near a pump, which are prone to error or require assumptions about at least one of the elevation of the sensing site relative to the joint, pressure drop across the tubing, pressure drop across one or more valves, or are only accurate at particular flow rates.

Thus, there is a need for improved fluid inflow and outflow sheaths that remedy the shortcomings of the prior art.

### SUMMARY

Accordingly, the present invention is directed to an improved arthroscopic surgery sheath and fluid flow system. An arthroscopic surgery sheath system according to an embodiment of the present invention comprises: a body for insertion of an instrument; an insertion portion coupled to the body; a valve coupled to the insertion portion; a tube coupled to the valve; and a microelectromechanical (MEM) sensor positioned inside the valve or the tube for measurement of at least one characteristic of a fluid inside of the tube; wherein the sensor is configured to transmit measurement information. The tube may be an inflow tube or an outflow tube. The sensor may be a pressure sensor or a temperature sensor and may sense both temperature and pressure. Optionally, the valve is a disposable stopcock valve. The sensor may be configured to wirelessly transmit measurement information.

In additional embodiments, the system has a receiver for receiving the measurement information transmitted by the sensor; a controller coupled to the receiver; and a fluid source coupled to the controller. The controller causes the fluid source to alter at least one characteristic of the fluid flowing through the tube based on the measurement information transmitted by the sensor. At least one of a display and an alarm may be coupled to the controller; and the controller may transmit fluid information to the display or activate the alarm based on the measurement information transmitted by the sensor. Optionally, the fluid source causes a high frequency pressure wave to be passed through the fluid inside the tube; and the sensor converts the pressure wave into energy.

In an additional embodiment, the present invention is directed to an arthroscopic surgery system comprising: a body for insertion of an instrument; a cannula coupled to the body; a sheath positioned inside the cannula and configured to create a lumen between the cannula and the sheath; a valve coupled to the cannula and communicating a fluid to the lumen; and a microelectromechanical sensor coupled to the cannula for measuring at least one characteristic of a fluid inside the lumen; wherein the sensor is configured to transmit measurement information.

The sensor may be positioned inside a wall of the cannula. The wall of the cannula may be thickened proximal to the sensor. The sensor may be a pressure sensor, a temperature sensor, or may sense both temperature and pressure. Optionally, the valve may be a disposable stopcock valve. Optionally, the cannula and the valve are disposable and the sheath is reusable. The sensor may be configured to wirelessly transmit measurement information.

In another embodiment, the present invention is directed to a fluid tube for arthroscopic surgery comprising: a body; a tube coupled to the body, the tube having a wall; a microelectromechanical sensor coupled to the wall of the tube for measuring at least one characteristic of a fluid inside the tube; and wherein the sensor is configured to transmit measurement information. The tube may be configured to receive fluid flowing out of a surgical site and the sensor configured to measure the temperature of the fluid. The sensor may be configured to wirelessly transmit measurement information.

### BRIEF DESCRIPTION OF THE DRAWINGS

The features, aspects and advantages of the present invention will become better understood with regard to the following description, appended claims and accompanying figures wherein:

FIG. 1 is a schematic diagram of an arthroscopic surgery system having an inflow/outflow sheath according to a first embodiment of the present invention;

FIG. 2 is a schematic diagram of an arthroscopic surgery system having an inflow/outflow sheath according to a second embodiment of the present invention; and

FIG. 3 is a schematic diagram of an arthroscopic surgery system having an inflow/outflow sheath according to a third embodiment of the present invention.

### DETAILED DESCRIPTION

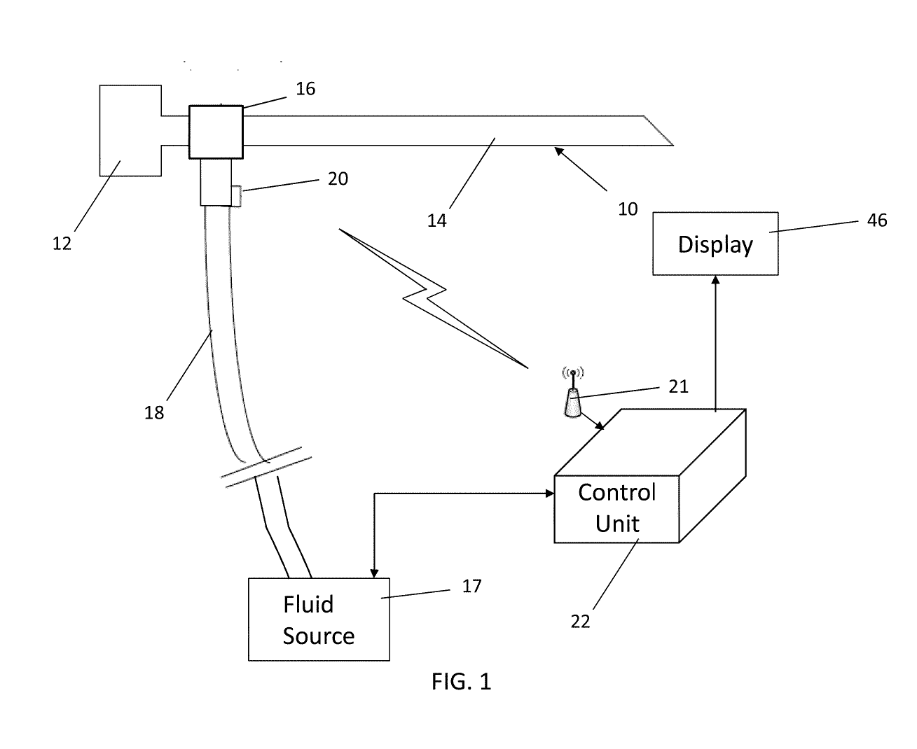

With reference to FIG. 1, the present invention, according to a first embodiment, is directed to an inflow/outflow sheath 10 having a sensor for use in arthroscopic surgery. The sheath 10 has a body 12 and an insertion portion 14. The sheath 10 has at least one disposable stopcock valve 16 for allowing fluid to enter the sheath 10. An inflow tube 18 is attached to the stopcock valve 16. During arthroscopic surgery, the inflow tube 18 is coupled to a pressurized fluid source 17, such as a pump. The fluid may also be fed to the inflow tube via gravity from a reservoir.

An operator may open the stopcock valve 16 to allow fluid to flow from the inflow tube 18, through the valve, and into the insertion portion 14. The fluid typically passes out a distal end of the insertion portion 14 to a surgical site. For example, during a surgical procedure on a joint, irrigating fluid is used to distend the joint to provide an adequate work space and in order to keep the joint free of debris while enhancing visibility during the procedure.

A sensor 20, such as a micro-electromechanical (MEM) sensor, is mounted inside of the disposable stopcock valve where the inflow tube 18 is inserted. Preferably, the sensor 20 is a wireless sensor that transmits sensed information to a receiver 21 in communication with or in a control unit 22. In an embodiment, the control unit 22 is coupled to the pressurized fluid source 17, and information from the sensor may be used by the control unit to adjust characteristics of the fluid passed to the inflow tube 18.

In an embodiment, the sensor 20 is a pressure sensor and the control unit 22 uses pressure information to maintain the fluid pressure within a predetermined range. In an additional embodiment, the sensor 20 is a temperature sensor and the control unit uses temperature information to maintain the fluid temperature within a predetermined range, such as by for example controlling the inflow and outflow of fluid. In an additional embodiment, the control unit 22 is coupled to a heating or cooling device in communication with the fluid and controls the heating or cooling device to maintain the fluid temperature within a predetermined range. In an additional embodiment, the valve 16 contains pressure and temperature sensors.

Sensors usable with the present invention include wireless capacitive sensors which are known in the art, such as for example the wireless capacitive sensors described in U.S. Pat. No. 6,926,670, entitled “Wireless MEMS Capacitive Sensor for Physiologic Parameter Measurement”, to Rich et al., the entire contents of which are hereby incorporated herein by reference.

Preferably, the sensor 20 is mounted inside of a wall of the valve 16. In an embodiment of the present invention, the valve wall contains a chamber or groove within which the sensor 20 is mounted so that the sensor may contact the fluid within the valve without impeding fluid flow. The valve wall may be thickened around the point where the sensor is mounted for strength and to provide additional mounting area for the sensor. Additionally the valve wall may have a sensor lumen with fluid passing into the sensor lumen and into contact with the sensor mounted in the wall. The sensor may be coupled to the valve wall using an adhesive, such as epoxy.

In an alternative embodiment, the sensor 20 is mounted in a hole in the valve wall such that the sensor communicates with the inside of the valve to contact fluid within the tubing and protrudes outside of the valve wall to allow for wired or wireless communication and for wired or wireless power to the sensor. In an alternative embodiment, the sensor is powered by a battery. In another embodiment, the sensor is powered remotely, such as by a high frequency pressure wave passed through the fluid itself. The mechanical energy superimposed on the fluid is then harvested at the sensor 20 by conversion of electrical power to operate both the sensor and wireless circuits. A piezoelectric material can harvest energy for this purpose.

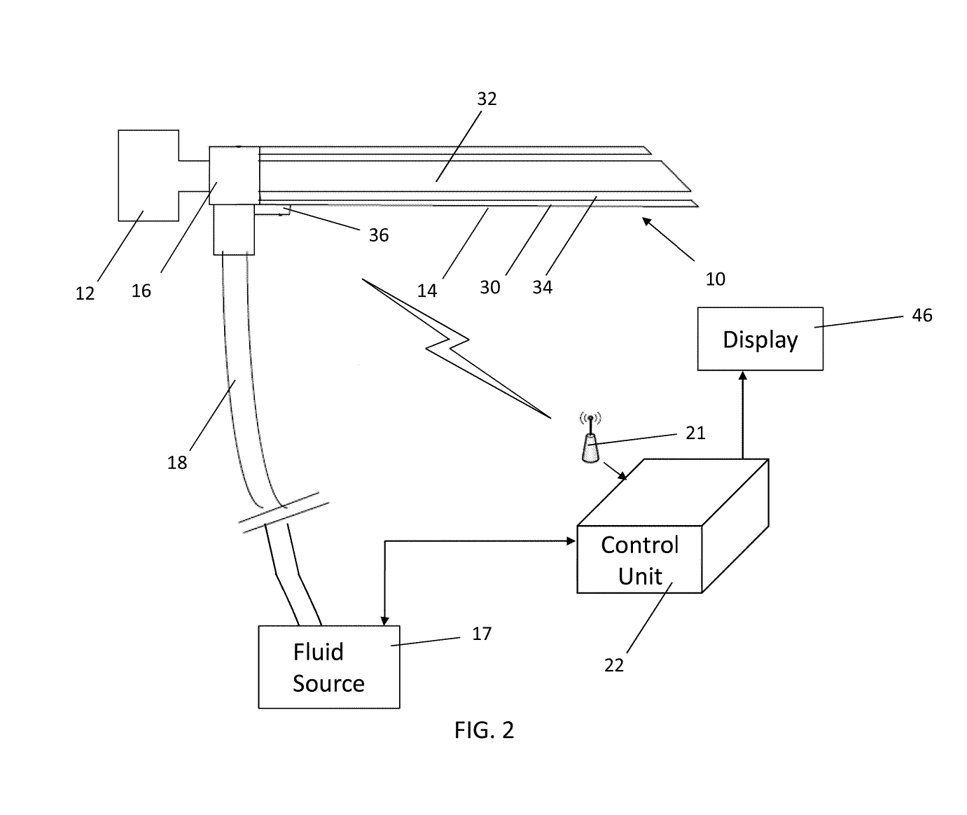

With reference to FIG. 2, the present invention according to a second embodiment is directed to an inflow/outflow sheath 10 having a sensor for use in arthroscopic surgery. The sheath 10 has a body 12 and an insertion portion 14. The sheath has a stopcock valve 16. Preferably, the stopcock valve 16 is disposable. As with the first embodiment, an inflow tube 18 is attached to the stopcock valve 16. During arthroscopic surgery, the inflow tube 18 is coupled to a pressurized fluid source 17 such as a pump. The insertion portion 14 has an outer cannula 30 and an inner sheath 32. A lumen 34 is formed between the outer cannula 30 and the inner reusable sheath 32.

An operator may open the stopcock valve 16 to allow fluid to flow from the inflow tube 18, through the valve, and into the lumen 34. A MEM Sensor 36 is affixed to the outer cannula to sense a characteristic of fluid in the lumen 34. Preferably, the sensor 36 is a wireless sensor that transmits sensed information to a receiver 21 in communication with or located in a control unit 22. In an embodiment, the sensor 36 uses pressure wave energy, such as sonic energy, for energy to communicate with the control unit 22. Information from the sensor 36 may be used to adjust characteristics of the fluid in the lumen 34.

In an embodiment, the sensor 36 is a pressure sensor and may be used to control the pressure of fluid at the surgical site. For example, the sensor 36 may be used to limit the flow of fluid through the inflow tube when the lumen pressure exceeds a predetermined amount. In an additional embodiment, the sensor 36 is a temperature sensor and the control unit 22 uses temperature information to maintain the fluid temperature within a predetermined range, such as by for example controlling the inflow and outflow of fluid. In an additional embodiment, the outer cannula 30 has pressure and temperature sensors.

Preferably, the outer cannula 30 is disposable and the inner sheath 32 is reusable. One advantage of a disposable outer cannula 30 incorporating the sensor 36 is that additional tubing is not required.

Preferably, the sensor 36 is mounted in a wall of the outer cannula 30. In an embodiment of the present invention, the outer cannula wall contains a chamber or groove within which the sensor 36 is mounted so that the sensor may contact the fluid within the lumen 34 without impeding fluid flow. The outer cannula wall may be thickened around the point where the sensor is mounted for strength and to provide additional mounting area for the sensor. Additionally the outer cannula wall may have a sensor lumen with fluid passing into the sensor lumen and into contact with the sensor mounted in the wall. The sensor may be coupled to the valve wall using an adhesive, such as epoxy. In an alternative embodiment, the sensor 36 is mounted in a hole in the outer cannula wall such that the sensor communicates with the inside of the lumen 34 to contact fluid within the lumen 34 and protrudes outside of the outer cannula 20 to allow for wired or wireless communication and for wired or wireless power to the sensor.

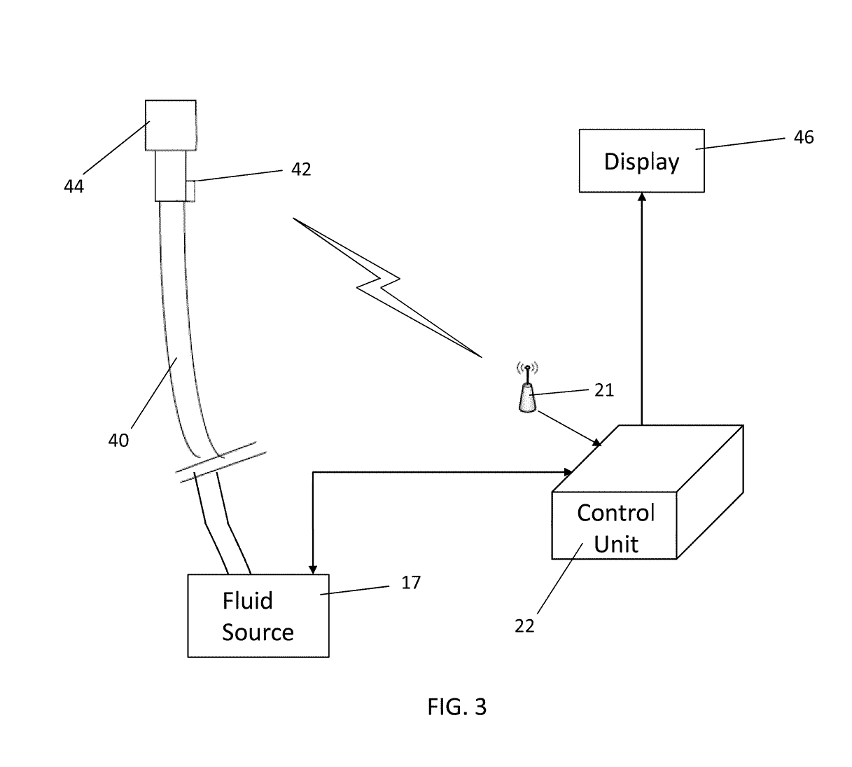

With reference to FIG. 3, the present invention, according to a third embodiment, is directed to an outflow tube 40 having a sensor 42 for use in arthroscopic surgery. The outflow tube 40 has a connector 44 for connecting the outflow tube to a sheath, cannula or other surgical instrument through which fluid is flowing out from a surgical site. Preferably, the sensor 42 is mounted in the outflow tube 40 in a position to sense a characteristic of fluid passing through the outflow tube. Preferably, the sensor 42 is a wireless sensor that transmits sensed information to a receiver 21 in communication with or in a control unit 22.

In an embodiment, the sensor 42 is a temperature sensor that measures the temperature of the fluid in the outflow tube 40 as an indicator of temperature at the surgical site. The control unit 22 may be coupled to a surgical monitor 46 and may display the sensed temperature on the surgical monitor. The control unit 22 may use the temperature information to alter fluid flow to heat or cool the surgical site or to trigger an alarm to a surgeon to limit temperature affecting activities.

For example, the temperature sensor 36 may allow for real time readings of saline temperature during ablation. During arthroscopy procedures it is common for users to utilize ablation devices to resect soft tissue. If the outflow of the saline is not controlled well, the temperature of the saline can rise to unsafe levels. By displaying the sensed temperature on the surgical monitor 46, the surgeon can know to limit the ablation if the temperature exceeds a certain predetermined threshold. Additionally, the fluid source 17, may be controlled based on temperature data to increase outflow if the saline temperature was approaching a dangerous level.

Preferably, the sensor 42 is inside of a wall of the outflow tube 40. In an embodiment of the present invention, the outflow tube wall contains a chamber or groove within which the sensor 42 is mounted so that the sensor may contact the fluid within the outflow tube 40 without impeding fluid flow. The outflow tube wall may be thickened around the point where the sensor 42 is mounted for strength and to provide additional mounting area for the sensor. Additionally the outflow tube wall may have a sensor lumen with fluid passing into the sensor lumen and into contact with the sensor 42 mounted in the wall. The sensor 42 may be coupled to the outflow tube wall using an adhesive, such as epoxy. In an alternative embodiment, the sensor 42 is mounted in a hole in the outflow tube wall such that the sensor communicates with the inside of the outflow tube 40 to contact fluid within the outflow tube and protrudes outside of the outflow tube wall to allow for wired or wireless communication and for wired or wireless power to the sensor.

The use of a MEM pressure sensor that wirelessly transmits sensor data allows for pressure to be sensed in tubing in a pump based system as well as in a gravity based system.

Users do not always disassemble a stopcock assembly from arthroscopy sheath systems. This can lead to difficulty in achieving sterilization and may lead to a buildup of corrosive material. A single use arthroscopy tubing set according to embodiments of the present invention that include a disposable stopcock assembly eliminates the possibility that the stopcock assembly will not be removed prior to sterile processing.

There is disclosed in the above description and the drawings, an arthroscopic surgery sheath and fluid flow system which fully and effectively overcomes the disadvantages associated with the prior art. However, it will be apparent that variations and modifications of the disclosed embodiments may be made without departing from the principles of the invention. The presentation of the preferred embodiments herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. §112.

What is claimed is:

1. An arthroscopic surgery sheath system comprising:
a body for insertion of an instrument;
an insertion portion coupled to the body;
a valve coupled to the insertion portion;
a tube coupled to the valve; and
a microelectromechanical sensor positioned inside the valve or the tube for measurement of at least one characteristic of a fluid inside of the tube;
wherein the sensor is configured to transmit measurement information.

2. The system of claim 1 wherein the tube is an inflow tube and the sensor is a pressure sensor.

3. The system of claim 1 wherein the tube is an outflow tube and the sensor is at least one of a pressure sensor and a temperature sensor.

4. The system of claim 1 wherein the valve is a disposable stopcock valve.

5. The system of claim 1 wherein the sensor is configured to wirelessly transmit measurement information.

6. The system of claim 5 further comprising:
a receiver for receiving the measurement information transmitted by the sensor;
a controller coupled to the receiver;
a fluid source coupled to the controller; and
wherein the controller causes the fluid source to alter at least one characteristic of the fluid flowing through the tube based on the measurement information transmitted by the sensor.

7. The system of claim 6 further comprising at least one of a display and an alarm coupled to the controller; and
wherein the controller transmits fluid information to the display or activates the alarm based on the measurement information transmitted by the sensor.

8. The system of claim 6 wherein the fluid source causes a high frequency pressure wave to be passed through the fluid inside the tube; and wherein the sensor converts the pressure wave into energy.

9. An arthroscopic surgery system comprising:
a body for insertion of an instrument;
a cannula coupled to the body;
a sheath positioned inside the cannula and configured to create a lumen between the cannula and the sheath;
a valve coupled to the cannula and communicating a fluid to the lumen; and
a microelectromechanical sensor coupled to the cannula for measuring at least one characteristic of a fluid inside the lumen;
wherein the sensor is configured to transmit measurement information.

10. The system of claim 9 wherein the sensor is positioned inside a wall of the cannula.

11. The system of claim 10 wherein the wall of the cannula is thickened proximal to the sensor.

12. The system of claim 9 wherein the sensor is at least one of a pressure sensor and a temperature sensor.

13. The system of claim 9 wherein the sensor is both a pressure sensor and a temperature sensor.

14. The system of claim 9 wherein the valve is a disposable stopcock valve.

15. The system of claim 9 wherein the cannula is disposable; the sheath is reusable and the valve is a disposable stopcock valve.

16. The system of claim 9 wherein the sensor is configured to wirelessly transmit measurement information.

17. A fluid tube for arthroscopic surgery comprising:
a body;
a tube coupled to the body, the tube having a wall;
a microelectromechanical sensor coupled to the wall of the tube for measuring at least one characteristic of a fluid inside the tube; and
wherein the sensor is configured to transmit measurement information.

18. The fluid tube of claim 17 wherein the tube is configured to receive fluid flowing out of a surgical site and the sensor is configured to measure the temperature of the fluid.

19. The fluid tube of claim 17 wherein the sensor is configured to wirelessly transmit measurement information.
